(19) United States
(12) Patent Application Publication
(10) Pub. No.: US 2025/0072878 A1
(43) Pub. Date: Mar. 6, 2025

(54) WIRE GUIDED MEDICAL DEVICES AND RELATED SYSTEMS AND METHODS OF USE

(71) Applicant: Boston Scientific Scimed, Inc., Maple Grove, MN (US)

(72) Inventors: Paul SMITH, Smithfield, RI (US); Scott E. BRECHBIEL, Burlington, MA (US); Andrew James SCHAUBHUT, Bolton, MA (US)

(73) Assignee: Boston Scientific Scimed, Inc., Maple Grove, MN (US)

(21) Appl. No.: 18/820,501

(22) Filed: Aug. 30, 2024

Related U.S. Application Data
(60) Provisional application No. 63/535,379, filed on Aug. 30, 2023.

(51) Int. Cl. A61B 10/04; A61B 10/02; A61M 25/09

(52) U.S. Cl. CPC A61B 10/04; A61B 2010/0216; A61M 2025/09183

(57) ABSTRACT

According to one aspect, a device may include a shaft having a brush at a distal portion of the shaft; a distal coupler configured to removably couple to a guidewire. The shaft is coupled to the distal coupler and the device is configured to slide in a distal direction or proximal direction along the guidewire.

### CROSS-REFERENCE TO RELATED APPLICATIONS

This application claims the benefit of priority of U.S. Provisional Patent Application No. 63/535,379, filed on Aug. 30, 2023, the entirety of which is incorporated herein by reference.

### TECHNICAL FIELD

Various aspects of the present disclosure relate generally to wire guided medical devices and related systems. More specifically, the present disclosure relates to medical devices and systems configured to removably couple to wires and related methods of use.

### BACKGROUND

During a medical procedure, for example an endoscopic retrograde cholangiopancreatography, or ERCP, procedure, an operator may utilize endoscopic techniques to diagnose biliary strictures. In some cases, a cytology brush is used to collect a sample for testing potentially malignant tissue. A guidewire is often used to position the cytology brush within, for example, the bile duct. The cytology brush is positioned at a target site, via a guidewire, to collect a sample of mucosal tissue layer off the surface of a lumen within the biliary track. However, in some cases, the tip of the cytology brush is forced off to the side of the guidewire by the brush's extending bristles, which can impede the brush from entering a stricture, injure other tissue, etc. In some cases, the cytology brush ends up terminating in a dilated region or pocket in front of a stricture. The current disclosure may solve one or more of these issues or other issues in the art.

### SUMMARY

Embodiments of the present disclosure relate to, among other things, medical devices and systems configured to couple to a guidewire, and related methods of use. Each of the embodiments disclosed herein may include one or more of the features described in connection with any of the other disclosed embodiments.

According to one aspect, a device may include a shaft having a brush at a distal portion of the shaft; a distal coupler configured to removably couple to a guidewire. The shaft is coupled to the distal coupler and the device is configured to slide in a distal direction or proximal direction along the guidewire.

In other aspects, the device may include one or more of the following features. The shaft may be configured to pivot relative to the distal coupler. The distal coupler may include a central channel, a proximal U-shaped portion, a distal U-shaped portion, and a slot between the proximal U-shaped portion and the distal U-shaped portion. The distal coupler may further comprise: (i) a middle portion positioned between the proximal U-shaped portion and the distal U-shaped portion, (ii) a first gap positioned between the middle portion and the distal U-shaped portion, and (iii) a second gap positioned between the middle portion and the proximal U-shaped portion. The middle portion may include an extending portion extending towards the slot. The distal coupler may be tapered from a proximal portion to a distal portion. The shaft may be coupled to the distal coupler via a ring-shaped hinge coupler. The distal U-shaped portion and the proximal U-shaped portion may be longitudinally aligned.

In other aspects, the device may include one or more of the following features. The distal coupler may be cylindrical and include a central lumen extending longitudinally entirely through the distal coupler, a channel may extend an entire longitudinal length of the distal coupler, and the channel may provide a lateral opening into the central lumen. The channel may extend between opposing surfaces of distal coupler, and the channel may have a width configured to allow a guidewire pass through the channel. The channel may extend transverse to a central longitudinal axis of the distal coupler. The channel may extend helically around the distal coupler. The distal coupler may be tapered and at least partially conical. The channel may be longitudinally aligned with a central longitudinal axis of the distal coupler. The distal coupler may include a main body, a first curved extension, a second curved extension, and first gap between the first curved extension and the second curved extension; and the first curved extension may form a second gap between the first curved extension and the main body. The second curved extension may form a third gap between the second curved extension and the main body; and the third gap may open into the first gap, the first gap may open into the second gap; and each of the first gap, the second gap, and the third gap may be sized to receive the guidewire. The distal coupler may include a first or proximal portion and a second or distal portion, and the first proximal portion may be removably coupled to the second distal portion via threads or grooves configured to receive threads. The distal coupler may include a proximal portion, a middle portion, a distal portion, and a hinge member extending between the proximal portion and the distal portion; and the hinge member may be configured to snap-fit or otherwise removably couple to middle portion and/or proximal portion and/or distal portion.

In other aspects, a device may comprise a shaft; and a distal coupler configured to removably couple to a guidewire. The shaft may be coupled to the distal coupler and the distal coupler comprises a central channel extending an entire longitudinal length of the distal coupler and including an opening facing a direction transverse from a central longitudinal axis of the distal coupler, a proximal U-shaped portion, a distal U-shaped portion, a middle portion extending between the proximal U-shaped portion and the distal U-shaped portion, and a hinge member rotatably coupled to at least one of the proximal U-shaped portion, the distal U-shaped portion, and the middle portion. The hinge member may be configured to transition from: (i) a position outside of the opening to (ii) a position extending across the opening, and the hinge member may be configured to snap-fit or otherwise removably couple to the middle portion and/or the proximal U-shaped portion and/or the distal U-shaped portion when positioned extending across the opening; and the device may be configured to slide in a distal direction or proximal direction along the guidewire.

In other aspects, a device may comprise a shaft; a distal coupler coupled to the shaft and configured to removably couple to a guidewire, wherein the distal coupler includes: a central channel, a proximal U-shaped portion, a distal U-shaped portion, a slot between the proximal U-shaped portion and the distal U-shaped portion, a middle portion positioned between the proximal U-shaped portion and the distal U-shaped portion, a first gap positioned between the middle portion and the distal U-shaped portion, and a second gap positioned between the middle portion and the proximal U-shaped portion. The device may be configured to slide in a distal direction or proximal direction along the guidewire. The middle portion may include an extending portion extending radially inward towards the slot.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a portion farthest away from a user when introducing a device into a patient. By contrast, the term “proximal” refers to a portion closest to the user when placing the device into the patient. Proximal and distal directions are labeled with arrows marked “P” and “D”, respectively, throughout the figures. Although endoscopes are referenced herein, reference to endoscopes or endoscopy should not be construed as limiting the possible applications of the disclosed aspects. For example, the disclosed aspects may be used with duodenoscopes, bronchoscopes, ureteroscopes, colonoscopes, catheters, diagnostic or therapeutic tools or devices, or other types of medical devices. Further, relative terms such as, for example, “about,” “substantially,” “approximately,” etc., are used to indicate a possible variation of ±10% in a stated numeric value or range.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 is a perspective view of an endoscope system and a magnified view of the distal end of the endoscope of the endoscope system, according to aspects of this disclosure.

FIG. 2 is a side view of a medical device coupled to a guidewire, according to aspects of this disclosure.

FIG. 3 is a magnified side view of the medical device of FIG. 2 coupled to a guidewire and shown in a different position, according to aspects of this disclosure.

FIG. 4 is a perspective view of a component of the medical device of FIG. 2, according to aspects of this disclosure.

FIG. 5 is a perspective view of another medical device, according to aspects of this disclosure.

FIG. 6 is a perspective view of a medical device coupled to a guidewire, according to aspects of this disclosure.

FIG. 7A is a perspective view of a medical device, according to aspects of this disclosure.

FIG. 7B is a perspective view of another medical device and a guidewire, according to aspects of this disclosure.

FIG. 8A is a side view of a medical device coupled to a guidewire, according to aspects of this disclosure.

FIG. 8B is a magnified view of the medical device of FIG. 8A coupled to the guidewire, according to aspects of this disclosure.

FIG. 9 is a side view of the medical device of FIG. 8A and a guidewire, with the medical device shown in two configurations, according to aspects of this disclosure.

FIG. 10A is a side view of a medical device coupled to a guidewire, according to aspects of this disclosure.

FIGS. 10B and 10C are magnified views of the medical device of FIG. 10A coupled to the guidewire, according to aspects of the present disclosure.

FIG. 11A is a side view of a medical device coupled to a guidewire, according to aspects of the present disclosure.

FIG. 11B is a magnified view of the medical device of FIG. 11A coupled to the guidewire, according to aspects of the present disclosure.

### DETAILED DESCRIPTION

Reference will now be made in detail to aspects of the present disclosure, examples of which are illustrated in the accompanying drawings. Wherever possible, the same or similar reference numbers will be used through the drawings to refer to the same or like parts.

Embodiments of this disclosure seek to improve movement of an medical device, such as a cytology brush, relative to a guidewire. Embodiments of this disclosure seek to improve a physician's ability to manipulate accessory devices, such as a cytology brush, coupled to a guidewire within a body lumen. Embodiments of this disclosure further seek to facilitate coupling, and specifically moveably coupling, an accessory device to a guidewire.

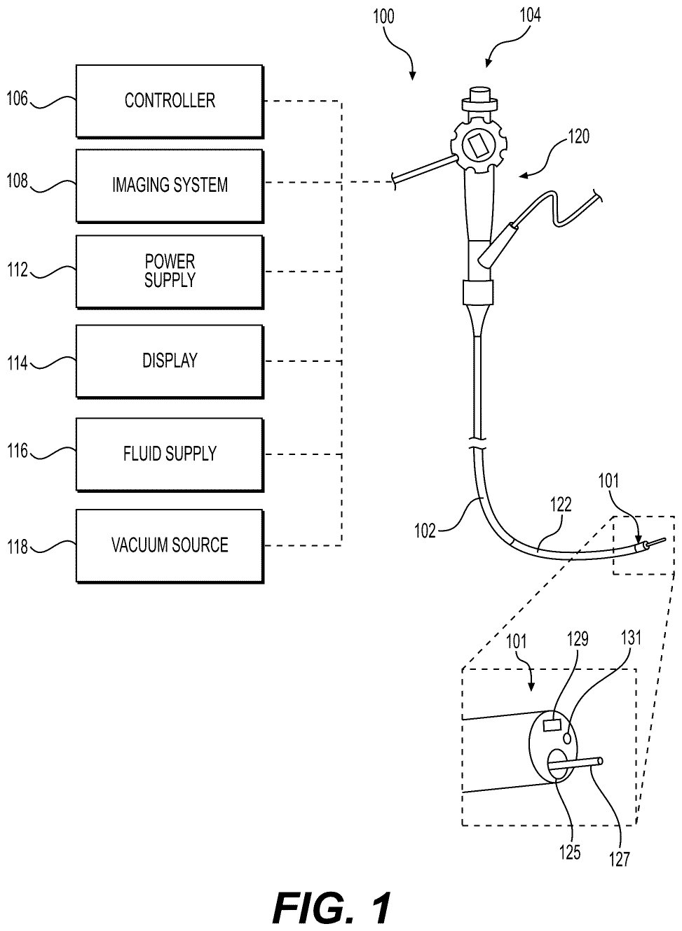

An exemplary endoscopy system 100 is shown in FIG. 1. Endoscopy system 100 may include an endoscope 104. Endoscope 104 may include a handle assembly 120 and a flexible tubular shaft 102. The flexibility of shaft 102 may be sufficient to allow shaft 102 to bend, to facilitate navigation of shaft 102 through a subject's tortuous anatomical passages. Shaft 102 may terminate at a distal tip 101. Shaft 102 may include an articulation section 122 for deflecting distal tip 101 in up, down, left, and/or right directions. In one example, articulation section 122 may provide for full retroflexion (e.g., rotation of distal tip 101 through an arc of 180 degrees) or only partial retroflexion (e.g., rotation of distal tip 101 through an arc of less than 180 degrees). Endoscope 104 also may include one or more lumens extending therethrough, and one or more openings in communication with the one or more lumens. For example, the one or more lumens may extend through handle assembly 120 and shaft 102, and the one or more openings may be on handle assembly 120 and distal tip 101. Endoscope 104 may be any suitable member for insertion into a patient's body, such as, e.g., an endoscope, a gastroscope, an ureteroscope, a nephroscope, a colonoscope, a hysteroscope, a ureteroscope, a bronchoscope, a cystoscope, a duodenoscope, a sheath, or a catheter.

One or more auxiliary devices may be operatively coupled to endoscope 104. Exemplary auxiliary devices may include a controller 106, an imaging system 108, a power supply 112, a display 114, a fluid supply 116, and/or a vacuum source 118, each of which is briefly described below. Controller 106 may include, for example, any electronic device capable of receiving, storing, processing, generating, and/or transmitting data according to instructions given by one or more programs. Controller 106 may be operatively coupled to, or part of, one or more of endoscope 104 and the other auxiliary devices, to control one or more aspects of their operation. Power supply 112 may include any suitable power source, and associated connectors (e.g., electrically-conductive wires), for supplying electronic components in the auxiliary devices and endoscope 104 with electrical power. Fluid supply 116 may include a reservoir, a medical irrigation bag, a pump, and any suitable connectors (e.g., tubing for fluidly coupling fluid supply 116 and endoscope 104). The pump may supply a flow of pressurized fluid to one or more of the lumens in endoscope 104, and the pressurized fluid flow may be emitted from distal tip 101 and/or used to inflate expandable components present at distal tip 101. Vacuum source 118 may provide suction or vacuum pressure to one or more lumens of the endoscope, and thereby provide a suction force to draw material toward and/or into endoscope 104, and/or to deflate expandable components.

Imaging system 108 may include imaging electronics to, for example, process signals received from an image sensor in endoscope 104, send signals for controlling the image sensor, adjust illumination levels of areas being viewed by the image sensor, and/or facilitate the display of image sensor data on display 114.

Distal tip 101 may include one or more image sensors 129 and one or more illuminators 131, shown in the magnified view of distal tip 101 in FIG. 1. One or more image sensors 129 may include a charge-coupled device image sensor, a complementary metal-oxide image semiconductor, or the like coupled to a cable or wire running through the shaft 102 of endoscope 104. One or more illuminators 131 may include light emitting diodes (LEDs) or the like.

A tool 127 may be inserted into a working channel or lumen 125 of endoscope 104, and tool 127 may exit out of the distal end of lumen 125. Tool 127 may include, for example, a brush, such as a wire brush, a guidewire, cutting or grasping forceps, a biopsy device, a snare loop, an injection needle, a cutting blade, scissors, a retractable basket, a retrieval device, an ablation and/or electrophysiology catheter, a stent placement device, a surgical stapling device, a balloon catheter, a laser-emitting device, and/or any other suitable therapeutic or diagnostic accessory device. As shown in the magnified view of distal tip 101, tool 127 has a smaller circumference about its longitudinal axis compared to the circumference about the longitudinal axis of lumen 125, and may include a smaller cross-sectional diameter as compared to the diameter of lumen 125. Aspects of this disclosure provide embodiments of medical devices that may facilitate movement of a tool, such as tool 127, to distal tip 101 and out of (e.g., distally out of) lumen 125 while coupled to a guidewire. Additionally or alternatively, aspects of this disclosure may facilitate navigation of tool 127 through tortuous body pathways, such as a bile duct. In some examples, any of the accessory devices described herein may be coupled to a guidewire and moved to a target site within a patient without the use of endoscope 104.

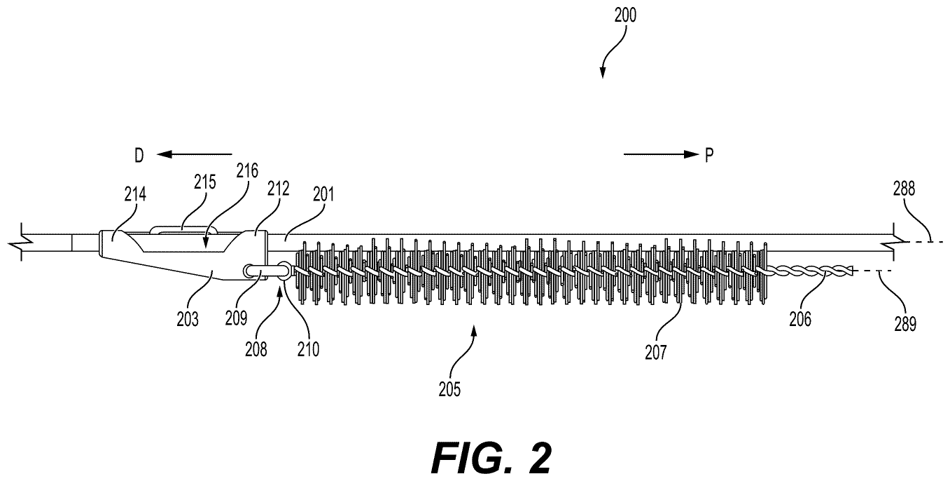

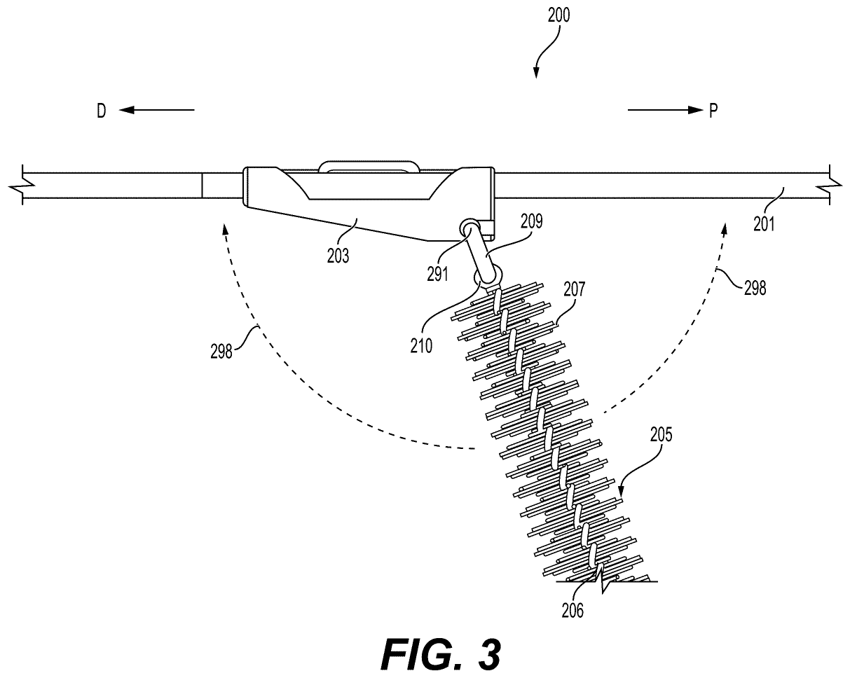

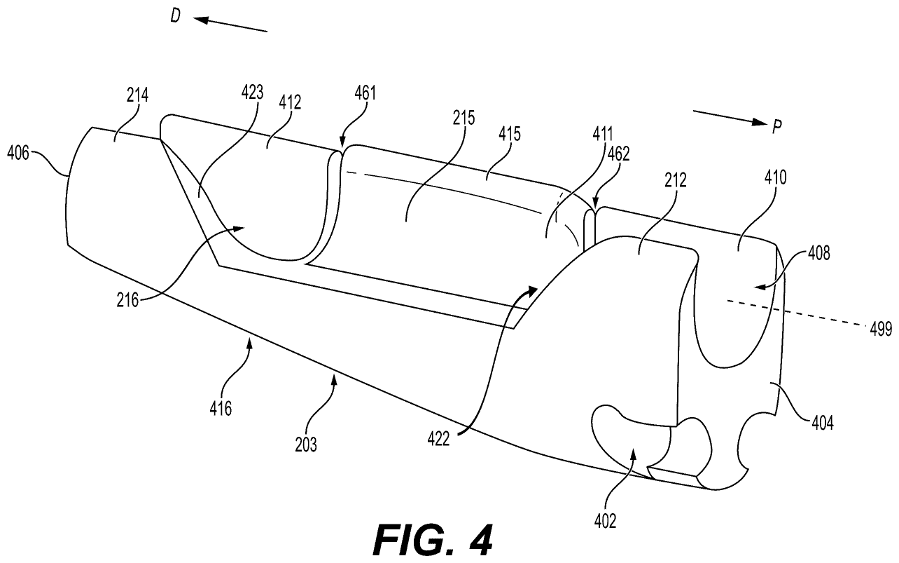

FIG. 2 illustrates a side view of a distal portion of a medical device 200 and a guidewire 201. Medical device 200 is shown moveably coupled to guidewire 201. Medical device 200 may include a distal coupler 203, a brush 205, and a shaft 206. Shaft 206 may be a wire, may be a wire including two or more wound or twisted wires, etc. In any of these aspects, shaft 206 may be rotatably coupled to a proximal portion 212 of distal coupler 203 via a hinge coupler 209. In some examples, hinge coupler 209 may be ring shaped. Hinge coupler 209 may extend through a lumen 402 (shown in FIG. 4) of distal coupler 203 and may also extend through a distal end portion 210 (shown in FIG. 3) of shaft 206. Distal coupler 203 may include a proximal portion 212, a distal portion 214, a middle portion 215, and a slot 216. Distal coupler 203 will be discussed in further detail below in relation to FIG. 4.

Brush 205 may comprise of a plurality of bristles 207 extending radially outward, relative to a central longitudinal axis 289 of shaft 206, from shaft 206. A central longitudinal axis 288 of guidewire 201 may be substantially parallel to central longitudinal axis 289 of shaft 206. Bristles 207 may be wire bristles, plastic bristles, polymer bristles, flexible bristles, rigid bristles, and/or any other type of bristles. In some examples, bristles 207 may include rounded end portions configured to be atraumatic. Brush 205 may be positioned at a distal portion of shaft 206, and may extend longitudinally along shaft 206 and extend circumferentially about longitudinal axis 289, and a plurality of bristles 207 may form a collective cylindrical shave with shaft 206 extending through a center of brush 205. In some examples, bristles 207 may be interwoven with shaft 206 to couple bristles 207 to shaft 206. For example, shaft 206 may be a wire with multiple wire strands, and bristles 207 may be sandwiched or otherwise positioned between at least two of the multiple wire strands of shaft 206. In some examples, each bristle 207 may be substantially the same length. In other examples, bristles 207 may have different lengths, and brush 205 may form shapes other than a cylinder, such as a spiral, a circle, a square, or any other suitable shape. Brush 205 may extend to a distal end portion 210 of shaft 206 and proximate to distal end portion 210. Brush 205 may be configured to pivot about distal end portion 210 relative to distal coupler 203 and/or guidewire 201, as discussed herein below.

As shown in FIG. 3, brush 205 may pivot about a pivot point 291 extending through hinge coupler 209, and brush 205 may be rotated relative to distal coupler 203 and guidewire 201. As shown by dotted-line arrows 298 in FIG. 3, brush 205 may be one hundred and eighty degrees rotatable relative to distal coupler 203, for example, from a position where brush 205 extends proximally from distal coupler 203 to a position where brush 205 extends distally from distal coupler 203. In some examples, brush 205 may pivot about distal end portion 210 and/or may pivot about pivot point 291. Distal end portion 210 may be circular on hinge coupler 209, and hinge coupler 209 may also be circular. Brush 205 may be moved distally by a user pushing shaft 206 distally and distal coupler 203 sliding along guidewire 201 in a distal direction, and may be moved proximally by a user pulling shaft 206 proximally and distal coupler 203 sliding along guidewire 201 in a proximal direction. In some examples, a user may pivot brush 205 relative to distal coupler 203 and guidewire 201 by pushing shaft 206 distally, for example, when distal coupler 203 abuts a portion of a patient that prevents distal coupler from sliding distally along guidewire 201. A user may also pivot brush 205 relative to distal coupler 203 and guidewire 201 by pulling shaft 206 proximally, for example after brush 205 has been moved distally and pivoted about pivot point 291 the user may pivot brush 205 in the opposite direction by pulling shaft 206 proximally. In some examples, a proximal portion of shaft 206 may be temporarily coupled to an additional tool, such as forceps or another medical device, to move shaft 206 proximally or distally. In other examples, a proximal portion of shaft 206 may extend to the user outside the body of the patient.

Guidewire 201 may be any guidewire known in the art, and, in some examples, may be a 0.018 inch, 0.021 inch, 0.025 inch, 0.035 inch, or any other suitable diameter guidewire. In some examples, distal coupler 203 may be configured to be releasably coupled to guidewire 201.

FIG. 4 illustrates a perspective view of distal coupler 203 removed or otherwise separated the other components of from medical device 200. As shown in FIG. 4, distal coupler 203 may include proximal portion 212, distal portion 214, middle portion 215, and slot 216. A U-shaped recess 408 may extend the entire length of distal coupler 203, from a proximalmost end 404 of distal coupler 203 to a distalmost end 406 of distal coupler 203. The U-shaped recess 408 may be formed by a radially-inward facing curved surface 410 of proximal portion 212, a radially-inward facing curved surface 411 of middle portion 215, and a radially-inward facing curved surface 412 of distal portion 214. A central longitudinal axis 499 may extend through U-shaped recess 408, and each of curved surfaces 410, 411, 412 may face central longitudinal axis 499. U-shaped recess 408 may be configured to slideably receive guidewire 201. For example, a user may push distal coupler 203 onto guidewire 201 such that guidewire 201 snaps, or is snap fit, into distal coupler 203. In some examples, U-shaped recess 408 may be narrower at the top portion of the U-shaped recess 408 proximate to the opening to the recess compared to the bottom portion of the U-shaped recess 408 on an opposite side of the opening of the U-shaped recess 408, which may facilitate coupling distal coupler 203 to guidewire 201.

Slot 216 may be formed between a distal-facing surface 422 of proximal portion 212 and a proximal-facing surface 423 of distal portion 214. Slot 216 may be configured to allow a user to access guidewire 201 to pull guidewire 201 out of distal coupler 203, in order to detach distal coupler 203 from guidewire 201. Middle portion 215 may include an extending portion 415, which extends towards slot 216 and is configured to facilitate coupling distal coupler 203 to guidewire 201. A first gap 461 may be positioned between middle portion 215 and distal portion 214, and a second gap 462 may be positioned between middle portion 215 and proximal portion 212. Gaps 461, 462 may be configured to allow middle portion 215, and in particular extending portion 415, to pivot relative to proximal portion 212 and distal portion 214 (e.g., radially outward relative to central longitudinal axis 499) to facilitate coupling distal coupler 203 to guidewire 201. In other examples, distal coupler 203 may not include gaps 461, 462.

Middle portion 215 of distal coupler 203 may be tapered such that proximalmost end 404 is larger than distalmost end 406, and distal coupler 203 may include an angled side surface 416 extending towards central longitudinal axis 499 as side surface 416 extends distally. Angled side surface 416 may be configured to be atraumatic, and may reduce trauma to tissue within a patient as distal coupler 203 is slid distally along guidewire 201 and/or through a body lumen. For example, angled side surface 416 may facilitate movement of medical device 200 through/across a stricture as medical device 200 (FIGS. 2 and 3) is pushed distally along guidewire 201, and may facilitate collecting a tissue sample with brush 205 within narrow body lumens.

Distal coupler 203 may include a proximal lumen 402 configured to receive hinge coupler 209, and hinge coupler 209 may be sized to allow hinge coupler 209 to be rotatable within proximal lumen 402. Proximal lumen 402 may be spaced from U-shaped recess 408, which may allow brush 205 to be spaced from guidewire 201 when medical device 200 is coupled to guidewire 201.

In operation, a user may first position guidewire 201 at a target site within a body of a patient. In some examples, the user may use endoscope system 100 to help position guidewire 201 at the target site. A user may then couple medical device 200 to a proximal portion of a guidewire 201, and push medical device 200 distally along guidewire 201 to the target site. In some examples, guidewire 201 may be positioned within lumen 125 of endoscope system 100, and the user may push medical device 200 through lumen 125 to the target site. In some examples, a user may push medical device 200 through a narrow body lumen, and the tapered shape of distal coupler 203 may facilitate moving medical device 200 through the narrow body lumen and/or may help to limit trauma to the patient's body tissue. Once at the target site, the user may move shaft 206 of medical device 200 proximally and/or distally to collect tissue in brush 205. In some examples, distal coupler 203 may abut a portion of the patient's body, such as a wall of a body lumen, which may prevent distal movement of distal coupler 203. When distal coupler 203 is abutting a portion of the patient's tissue and distal movement is limited, the user may push and/or pull shaft 206 distally and/or proximally to pivot brush 205 about pivot point 291. Shaft 206 may be flexible such that only a distal portion of shaft 206 pivots about pivot point 291. By pivoting brush 205 about pivot point 291, the user may collect additional patient tissue using brush 205. In some examples, the user may visualize at least a portion of the target site using endoscope system 100 (e.g., with image sensor(s) 129 and/or illuminator(s) 131). Once tissue has been collected by brush 205, the user may then pull medical device 200 proximally to remove medical device 200 from the patient, and, in some examples, collect a tissue sample from brush 205 once brush 205 is removed from the patient.

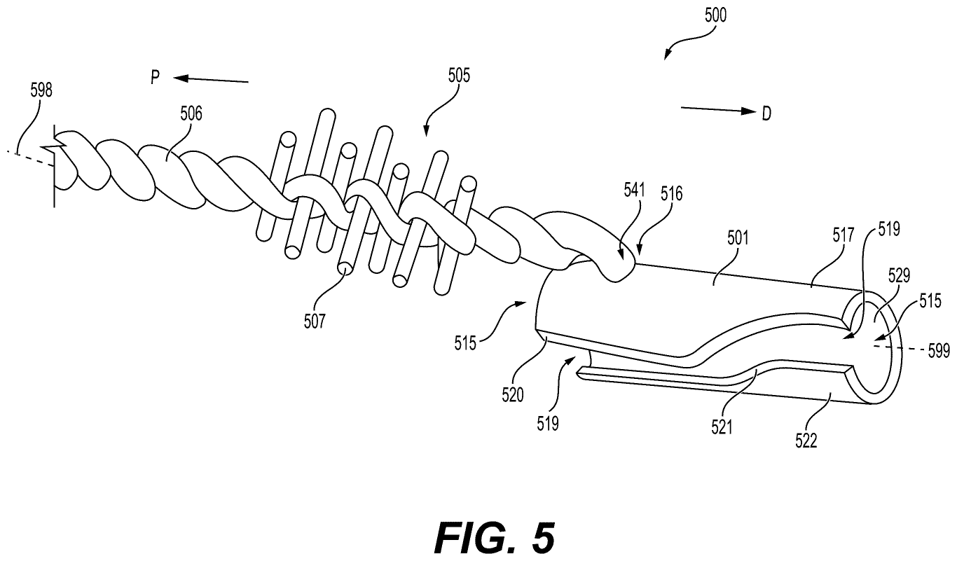

FIG. 5 illustrates a distal portion of an alternative embodiment of a medical device 500 including a distal coupler 501, a brush 505, and a shaft 506. Medical device 500 may operate substantially the same way as medical device 200. However, brush 505 may be fixedly coupled to distal coupler 501, and shaft 506 may not be configured to pivot relative to distal coupler 501. As shown in FIG. 5, shaft 506 may be fixedly coupled to distal coupler 501 at a proximal portion 516 of distal coupler 501. Bristles 507 of brush 505 may be interwoven with shaft 506, such that each bristle 507 extends radially outward from a central longitudinal axis 598 of shaft 506. Distal coupler 501 may be configured to removably couple to a guidewire (e.g., guidewire 201 in FIGS. 2 and 3).

Distal coupler 501 may be cylindrical and/or may be tapered in the proximal-distal direction such that a distal portion 517 of distal coupler 501 may have a smaller diameter about longitudinal axis 599 than proximal portion 516 of distal coupler 501. In other examples, distal coupler 501 may be partially cylindrical and may have a uniform diameter about central longitudinal axis 599. A distalmost end 541 of shaft 506 may be fixedly coupled to a radially-outward facing surface 522 of distal coupler 501. A central lumen 515 may extend longitudinally through a central portion of distal coupler 501, and central lumen 515 may be configured to receive guidewire 201. Central lumen 515 may have a diameter larger than the diameter of guidewire 201, for example, allowing for distal coupler 502 to slide (e.g., distally and/or proximally) along guidewire 201. A radially-inward facing surface 529 of distal coupler 501 may form central lumen 515, and surface 529 may extend the entire longitudinal length of distal coupler 501. A channel 519 may extend the entire longitudinal length of distal coupler 501, and may provide a lateral opening, or an opening transverse from central longitudinal axis 599, into lumen 515. Channel 519 may extend between opposing surfaces 520, 521 of distal coupler 501, and channel 519 may have a width, or a distance between surface 520 and surface 521, larger than the diameter of guidewire 201 to allow guidewire 201 to pass through channel 519. As shown in FIG. 5, channel 519 may extend longitudinally transverse from central longitudinal axis 599. A user may move guidewire 201 through channel 519 and into lumen 515, then rotate distal coupler 501 relative to guidewire 201 to align central longitudinal axis 599 with a longitudinal axis of guidewire 201. Then a user may translate distal coupler 501 proximally and distally along guidewire 201 without distal coupler 501 uncoupling from guidewire 201. Distal coupler 501 may be flexible to expand and contract channel 519, for example to move distal coupler 501 around guidewire 201, and then channel 519 may reduce in size after coupling to guidewire 201 to help retain guidewire 201 in distal coupler 501.

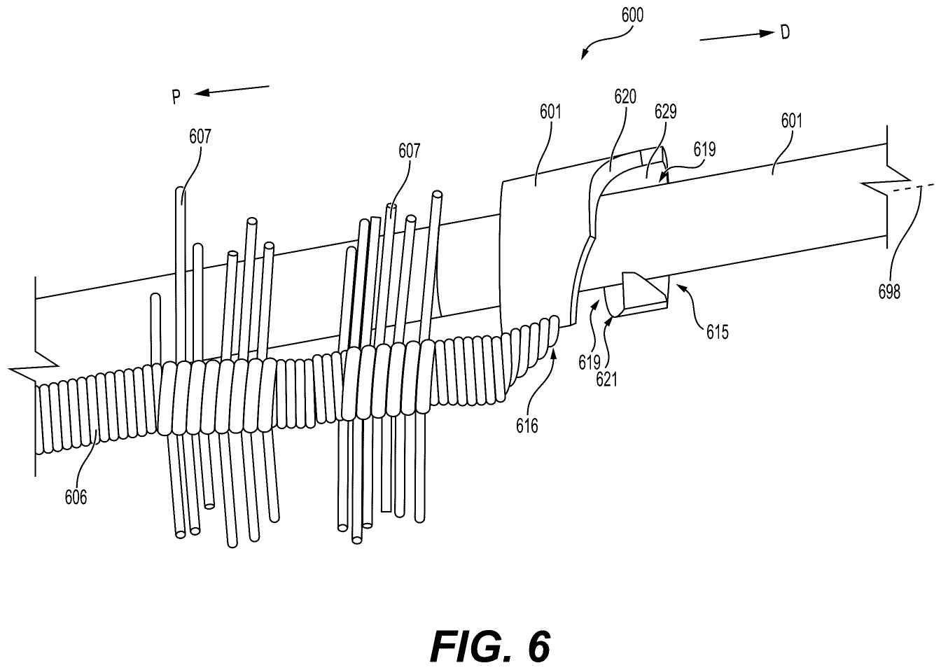

FIG. 6 illustrates a side view of another embodiment of a medical device 600 coupled to a guidewire 601. Medical device 600 may include any of the features described herein with relation to medical devices 200, 500. Medical device 600 includes a shaft 606, a distal coupler 601, and two brushes 607, 608. First brush 607 may be longitudinally spaced from second brush 608, and each brush 607, 608 may be coupled to shaft 606. Shaft 606 may be fixedly coupled to distal coupler 601 at a proximal portion 616 of distal coupler 601. Distal coupler 601 may include a central lumen 615 extending longitudinally through distal coupler 601 and formed by a radially-inward facing, relative to central longitudinal axis 698, curved surface 629. A channel 619 may extend the entire longitudinal length of distal coupler 601, and may provide a lateral opening, or an opening transverse from central longitudinal axis 599, into lumen 615. Channel 619 may extend between opposing surfaces 620, 621 of distal coupler 601, and channel 619 may have a width, or a distance between surface 620 and surface 621, larger than the diameter of guidewire 601 to allow guidewire 601 to pass through channel 619. As shown in FIG. 6, channel 619 may extend longitudinally transverse from central longitudinal axis 698, such that a user may move guidewire 691 through channel 619 and into lumen 615, then rotate distal coupler 601 relative to guidewire 691 to align central longitudinal axis 698 with a longitudinal axis of guidewire 691. A user may then translate distal coupler 601 proximally and distally along guidewire 691 without distal coupler 601 uncoupling from guidewire 691.

Channel 619 may be helical shaped and may corkscrew through at least a portion of distal coupler 601. In some examples, distal coupler 601 may be flexible to allow a user to stretch channel 619 wider to accommodate guidewire 691, and the width of channel 619 in an unstretched configuration may be less than the diameter of guidewire 691, helping to retain guidewire 691. The helical shape of channel 619 may facilitate maintaining the coupling between medical device 600 and guidewire 691 while medical device 600 is translated proximally and distally along guidewire 691.

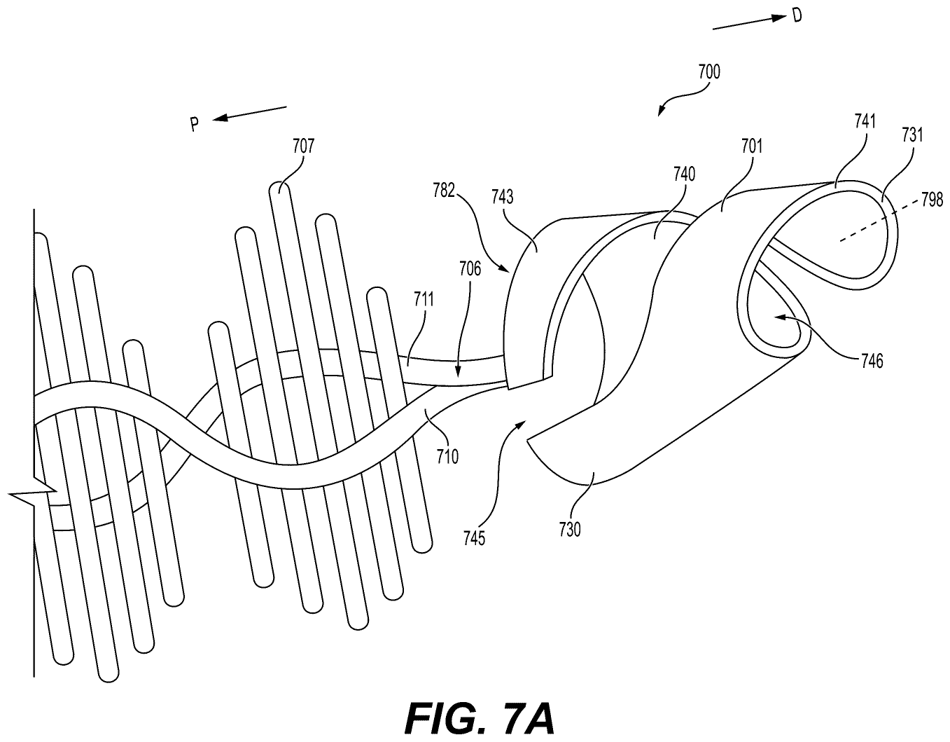

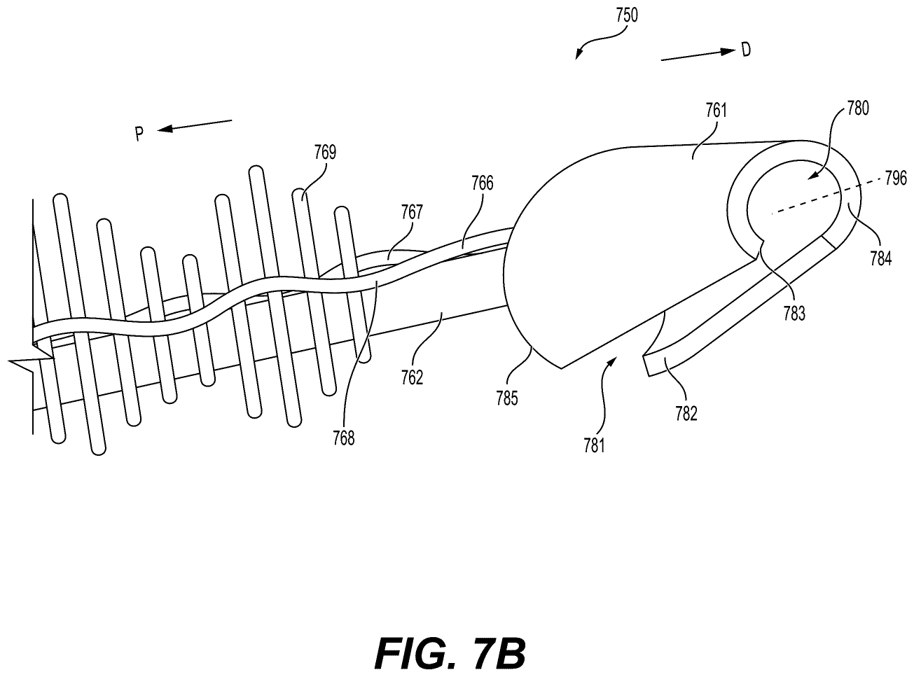

FIG. 7A illustrates an alternative embodiment of a medical device 700 including a shaft 706, distal coupler 701, and at least one brush 707. Medical device 700 may have any of the features discussed herein with relation to medical devices 200, 500, 600. Shaft 706 may include two wound wires 710, 711, and brush 707 may be positioned between wires 710, 711 to couple brush 707 to shaft 706. Distal coupler 701 may include a central lumen 746. Central lumen 746 may extend longitudinally through distal coupler 701 and may be formed by a radially-inward facing surface 740, relative to central longitudinal axis 798, of distal coupler 701. A channel 745 may extend through distal coupler 701 from a distalmost end to a proximalmost end of distal coupler 701. Channel 745 may be helical and may be positioned between an edge surface 731 of distal coupler 701. Distal coupler 701 may be tapered such that the diameter of distal coupler 701 (and the diameter of central lumen 746) becomes smaller as distal coupler 701 extends distally. The smallest diameter portion of central lumen 746 may be sized to receive guidewire 201, and the conical shape of distal coupler 701 may facilitate coupling distal coupler 701 to guidewire 201. Also, the conical shape of distal coupler 701 may help to reduce trauma to tissue as distal coupler 701 is pushed distally through a body lumen of a patient. Shaft 706 may be fixedly coupled to a proximal-facing surface 782 of distal coupler 701 at a proximalmost end of distal coupler 701.

FIG. 7B illustrates another embodiment of a medical device 750 including a shaft 766, a brush 769, and a distal coupler 761. Medical device 750 may have any of the features described herein in relation to medical devices 200, 500, 600, 700. Medical device 750 is shown with a portion of medical device 750 positioned over guidewire 762. Distal coupler 761 may be at least partially conical (e.g., forming a truncated cone) and may include a central lumen 780 extending longitudinally through distal coupler 761. Shaft 766 may be coupled to a proximalmost end 785 of distal coupler 761, and shaft 766 may include wires 767, 768 wound around each other. Brush 769 may extend radially-outward from shaft 766. A channel 781 may extend longitudinally from the proximalmost end 785 of distal coupler 761 to a distalmost end 784 of distal coupler 761. Channel 781 may be substantially linear and may extend in a proximal-distal direction. In these aspects, distal coupler 761 may form a truncated cone, with a longitudinally extending channel. The smallest diameter portion of lumen 780 (e.g., at distalmost end 784 of distal coupler 761) may be sized to receive guidewire 762. Central lumen 780 may extend along a central longitudinal axis 796 of distal coupler 761, and the conical shape of distal coupler 761 may facilitate coupling distal coupler 761 to guidewire 762. Channel 781 may be sized to allow guidewire 762 to pass through channel 781 and into lumen 780. Opposing planar surfaces 782, 783 may form channel 781, and each surface 782, 783 may longitudinally extend in a proximal-distal direction. The straight or linear shape of channel 781 longitudinally aligned with central longitudinal axis 796 may facilitate quickly coupling distal coupler 761 to guidewire 762.

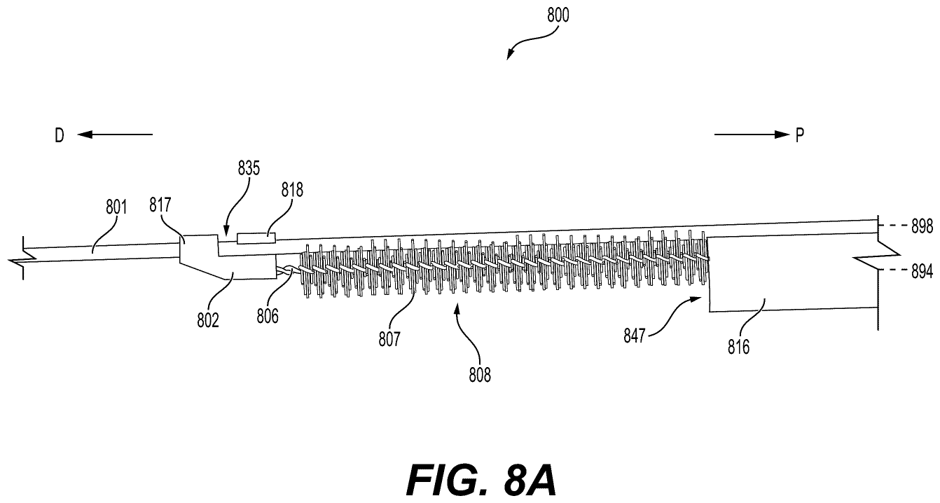

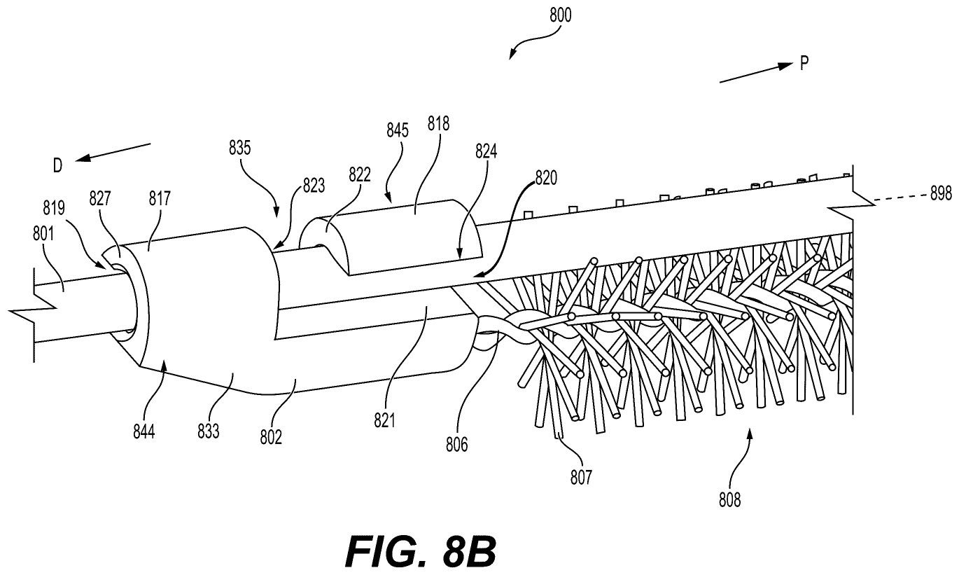

FIGS. 8A and 8B illustrates another embodiment of a medical device 800 coupled to a guidewire 801. As shown in FIG. 8A, medical device 800 may include a distal coupler 802, a shaft 806, and a brush 808. Medical device 800 may include any of the features described herein with relation to medical devices 200, 500, 600, 700, 750. A sheath 816 may be positioned over at least a portion of shaft 806, and shaft 806 may extend through a longitudinal lumen 847 of sheath 816. Lumen 847 may be sized to allow shaft 806 and brush 808 to move through sheath 816 in a proximal or distal direction as a user moves sheath 816 relative to shaft 806, or vice versa. In other examples, medical device 800 may not include sheath 816. Shaft 806 may be fixedly coupled to distal coupler 802 at a proximalmost end of distal coupler 802. A longitudinal axis 894 of sheath 816 may be substantially parallel to a longitudinal axis 898 of guidewire 801 during operation of medical device 800. Brush 808 may contain a plurality of bristles extending radially-outward from shaft 806.

Distal coupler 802 may include a first curved extension 817 positioned at a distal portion of distal coupler 802, and a second curved extension 818 positioned at a proximal portion of distal coupler 802. Referring to FIG. 8B showing a magnified view of medical device 800, first curved extension 817 may include a portion of a distalmost front face 827 of distal coupler 802 and may be longitudinally spaced from second curved extension 818. Each of first curved extension 817 and second curved extension 818 may extend from a main body portion 833 of distal coupler 802. Each of first curved extension 817 and second curved extension 818 may include a curved, radially-inward facing surface configured to abut and/or align with guidewire 801, for example, to help movably retain guidewire 801. First curved extension 817 may extend from a first side 844 of main body portion 833, and second curved extension 818 may extend from a second side 845 of main body portion 833. The first side 844 may be divided from the second side 845 by a longitudinal axis of main body portion 833 of distal coupler 802. First curved extension 817 may be longitudinally aligned with second curved extension 818, such that when guidewire 801 is positioned within each of first curved extension 817 and second curved extension 818, guidewire 801 extends along a central longitudinal axis 898 of guidewire 801 and is not curved. Although first curved extension 817 and second curved extension 818 are shown curved, they are not so limited and may be L-shaped or any other shape to facilitate coupling to guidewire 801.

A first gap 835 may be formed between first curved extension 817 and second curved extension 818, and gap 835 may be wide enough to receive guidewire 801. First curved extension 817 may form a second gap 819 between first curved extension 817 and main body portion 833. Second curved extension 818 may form a third gap 820 between second curved extension 818 and main body portion 833. Third gap 820 may open into first gap 835, and first gap 835 may open into second gap 819 such that each of first gap 835, second gap 819, and third gap 820 are fluidly connected. Each of first gap 835, second gap 819, and third gap 820 may be sized to receive guidewire 801. Third gap 820 may be positioned between a first planar surface 821 of main body portion 833 and a second surface 824 of second curved extension 818, and second surface 824 may be a portion of the curved, radially-inward facing surface of second curved extension 818. First gap 835 may extend completely across, or in a direction transverse a longitudinal axis of main body portion 833, and each of second gap 819 and third gap 820 may extend only partially across main body portion 833. As shown in FIG. 8B, shaft 806 may be coupled to a proximal end of main body portion 833.

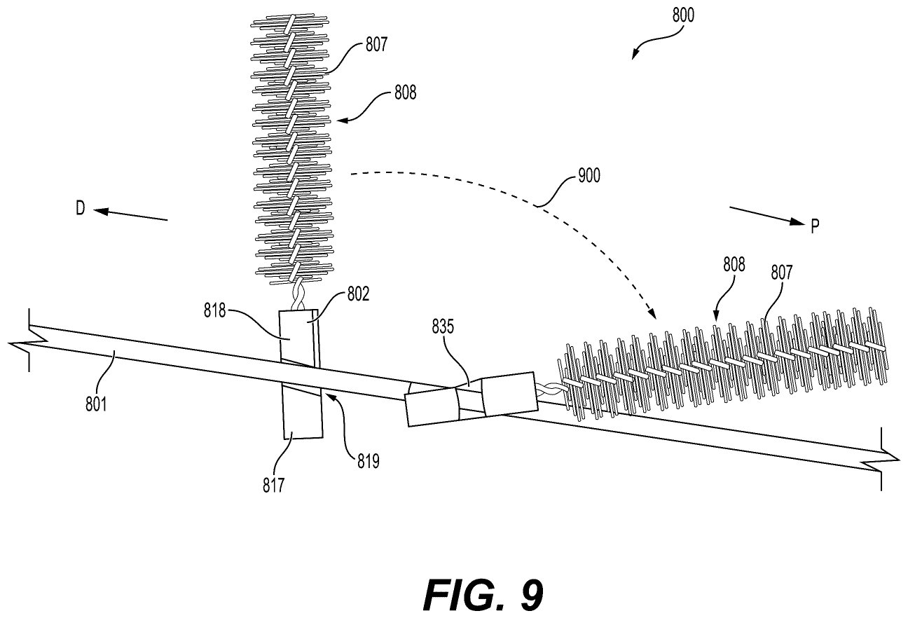

FIG. 9 illustrates a top view of medical device 800 being coupled to guidewire 801, and medical device 800 is shown twice (i.e., in two positions) for demonstration purposes. To couple medical device 800 to guidewire 801, a user may align first gap 835 with guidewire 801 and position guidewire 801 within first gap 835. Once guidewire is positioned completely within first gap 835, the user may rotate medical device 800 (shown by dotted-line arrow 900 in FIG. 9) to move guidewire 801 within second gap 819 and third gap 820. Once distal coupler 802 is rotated approximately ninety degrees, distal coupler 802 may be longitudinally aligned with guidewire 801, and medical device 800 may be removably coupled to guidewire 801. Once the user has rotated guidewire 801 into second gap 819 and third gap 820 and guidewire 801 extends longitudinally through distal coupler 802, the user may move medical device 800 distally or proximally along guidewire 801 without medical device 800 uncoupling from guidewire 801. In some examples, the user may then cover brush 808 and distal coupler 802 with sheath 816 (FIG. 8A) to help prevent brush 808 from abutting tissue, and potentially causing unwanted trauma, within a patient while the user positioned medical device 800 at a target site.

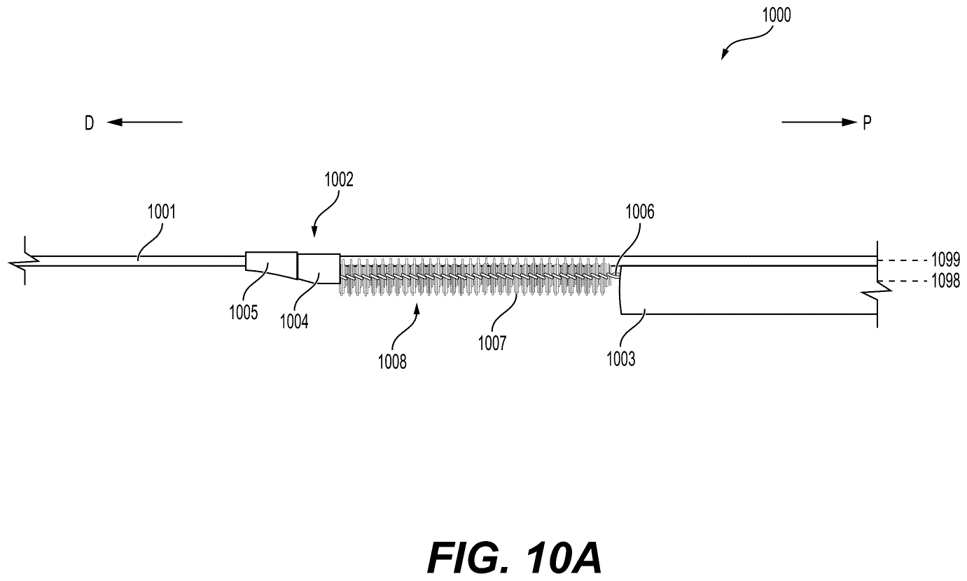

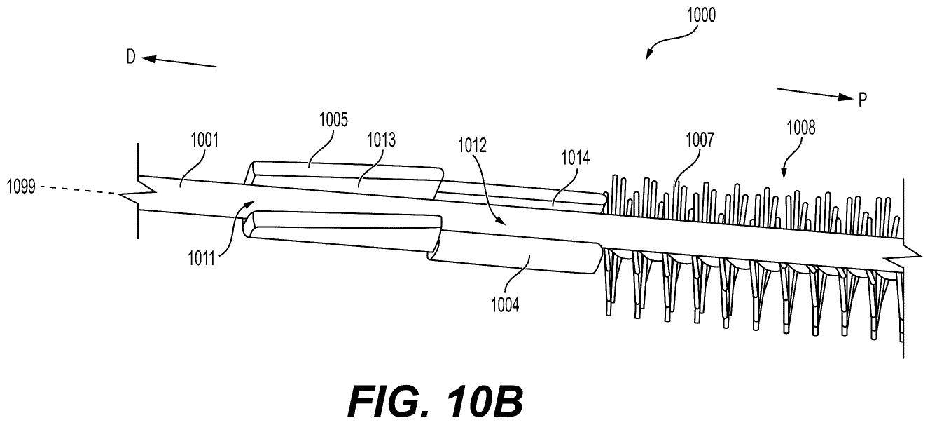

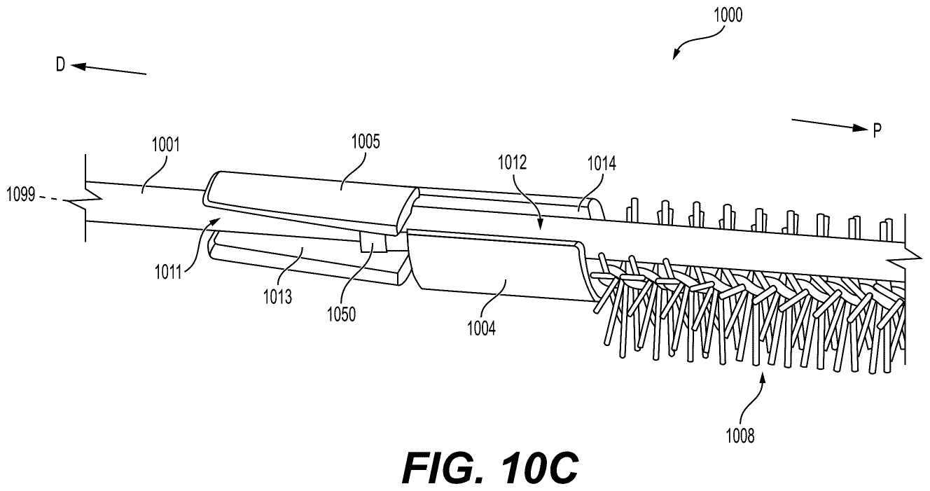

FIGS. 10A-10C illustrate another embodiment of a medical device 1000 coupled to a guidewire 1001. As shown in FIG. 10A, medical device 1000 may include a distal coupler 1002, a shaft 1006, and a brush 1008 including bristles 1007. Medical device 1000 may include any of the features described herein with relation to medical devices 200, 500, 600, 800. In some examples, medical device 1000 may include a sheath 1003. FIGS. 10B and 10C illustrate magnified views of medical device 1000. FIG. 10B shows distal coupler 1002 in a first or open configuration, and FIG. 10C shows distal coupler 1002 in a second or closed configuration. Distal coupler 1002 may include a first or proximal portion 1004 and a second or distal portion 1005, and first proximal portion 1004 may be rotatably coupled to second distal portion 1005. Each of proximal portion 1004 and distal portion 1005 may be U-shaped and configured to receive guidewire 1001. Distal portion 1005 may include a radially-inward facing curved surface 1013 and a first gap 1011 formed between the curved surface 1013. Proximal portion 1004 may include a radially-inward facing curved surface 1014 and a second gap 1012 formed between the curved surface 1014.

In the open position shown in FIG. 10B, proximal portion 1004 may be longitudinally aligned with distal portion 1005, such that first gap 1011 is longitudinally aligned with second gap 1012, and guidewire 1001 may be positioned with first gap 1011 and second gap 1012. In the open position, distal coupler 1002 may be moved transverse to a central longitudinal axis 1099 of guidewire 1001 to position guidewire 1001 within first gap 1011 and second gap 1012, without having to slide distal coupler 1002 over a proximal end or distal end of guidewire 1001. A user may transition medical device 1000 from the open position (shown in FIG. 10B) to the closed position (shown in FIG. 10C) by rotating distal portion 1005 relative to proximal portion 1004, for example, about a longitudinal axis of distal coupler 1002. As shown in FIG. 10C, the closed configuration of medical device 1000 removably couples distal coupler 1002 to guidewire 1001 when guidewire 1001 is positioned within first gap 1011 and second gap 1012. In some examples, distal portion 1005 may be configured to rotate ninety degrees relative to proximal portion 1004 from the open position to the closed position. In other examples, distal portion 1005 may be configured to rotate forty-five degrees, one hundred and twenty degrees, or any other amount of rotation greater than ten degrees relative to proximal portion 1004 to transition from the open position to the closed position. Distal portion 1005 may be tightened relative to the proximal portion 1004, such that when distal portion 1005 is released (e.g., by the user) distal portion 1005 does not move relative to proximal portion 1004. Once distal coupler 1002 is coupled to guidewire 1001 and in a closed configuration, a user may translate distal coupler 1002 proximally or distally along guidewire 1001 by pushing or pulling shaft 1006 (FIG. 10A).

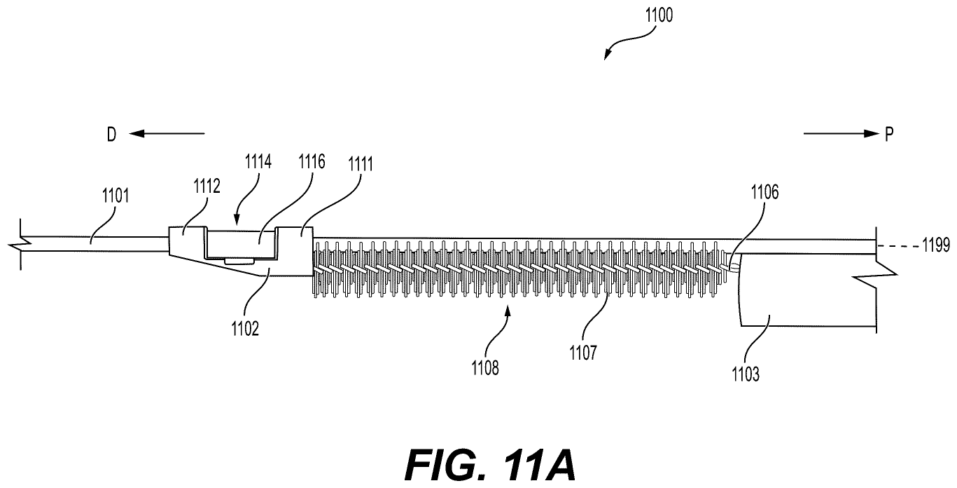

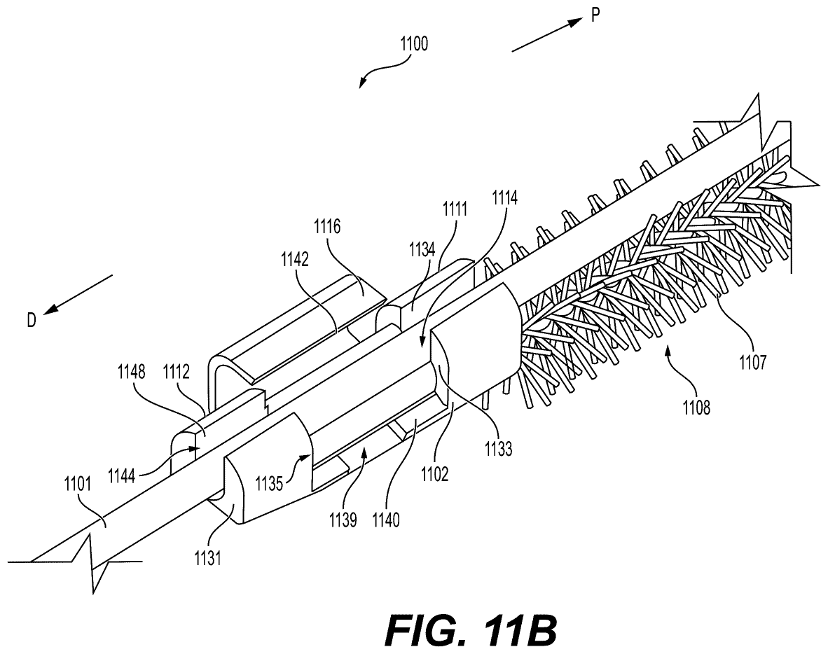

FIGS. 11A and 11B illustrate another embodiment of a medical device 1100 coupled to a guidewire 1101. As shown in FIG. 11A, medical device 1100 may include a distal coupler 1102, a shaft 1106, and a brush 1108 including bristles 1107. Medical device 1100 may include any of the features described herein with relation to medical devices 200, 500, 600, 800, 1000. In some examples, medical device 1100 may include a sheath 1103. FIG. 11A illustrates a first or closed configuration of distal coupler 1102, and FIG. 11B illustrates a magnified view of medical device 1100 and shows distal coupler 1102 in a second or open configuration. Shaft 1106 may be fixedly coupled to a proximal portion 1111 of distal coupler 1102, and distal coupler 1102 may be tapered such that proximal portion 1111 is larger than a distal portion 1112 of distal coupler 1102. A hinge member 1116 of distal coupler 1102 may be positioned within a gap 1114 between proximal portion 1111 and distal portion 1112 of distal coupler 1102. Hinge member 1116 may extend around a central longitudinal axis 1199 of guidewire 1101 when guidewire 1101 is coupled to distal coupler 1102.

Referring to FIG. 11B, a central channel 1144 may longitudinally extend through the entire length of distal coupler 1102, and may be configured to received guidewire 1101. A curved, radially-inward facing surface 1148 of distal portion 1112 may form a portion of central channel 1144; a curved, radially-inward facing surface 1134 of proximal portion 1111 may form another portion of central channel 1144; and central channel 1144 may extend to a distal front face 1131 of distal coupler 1102. Hinge member 1116 may be U-shaped, L-shaped, or any other shape configured to extend at least partially around guidewire 1101 when guidewire 1101 is positioned within central channel 1144. Hinge member 1116 may include a longitudinal edge surface 1142 substantially parallel to central longitudinal axis 1199 and extending the length of gap 1114. A middle portion 1140 of distal coupler 1102, which may connect the proximal portion 1111 to the distal portion 1112, may include a recess 1139. Recess 1139 may be configured to facilitate accessing longitudinal edge surface 1142 with a user's finger(s) to move hinge member 1116 from a closed configuration (shown in FIG. 11A) to an open configuration (shown in FIG. 11B). Hinge member 1116 may be configured to snap-fit or otherwise removably couple to middle portion 1140 and/or proximal portion 1111 and/or distal portion 1112.

To couple medical device 1100 to guidewire 1101, a user may first position guidewire 1101 within central channel 1144 while medical device 1100 is in an open configuration with hinge member 1116 pivoted away from middle portion 1140. Once guidewire 1101 is positioned within central channel 1144, the user may move/pivot hinge member 1116 towards middle portion 1140 and snap-fit hinge member 1116 to the rest of the body of distal coupler 1102 (e.g. middle portion 1140 and/or proximal portion 1111 and/or distal portion 1112). Hinge member 1116 may help to prevent guidewire 1101 from sliding out of central channel 1144 and/or uncoupling from distal coupler 1102 during operation. Central channel 1144 may be sized to allow guidewire 1101 to move proximally and/or distally through central channel 1144, and limit lateral movement or movement transverse to central longitudinal axis 1199.

In any of the above-described embodiments, a medical device 200, 500, 600, 700, 750, 800, 1000, 1100 may include one or more components that are metallic, a polymer, machined, formed, stamped, insert molded, or any combination thereof. Any of the above-described embodiments, a medical device 200, 500, 600, 800, 1000, 1100 may be side-loaded onto a guidewire during an operation, without accessing a proximalmost end or distalmost end of the guidewire. In any of the above-described embodiments, a medical device 200, 500, 600, 800, 1000, 1100 may include a distal coupler with a closed loop coupling assembly requiring a guidewire to be threaded through the medical device 200, 500, 600, 800, 1000, 1100. In any of the above-described embodiments, a medical device 200, 500, 600, 800, 1000, 1100 may include a clasping mechanism for attachment onto a guidewire configured to be pressed by fingers or through a separate tool to permanently deform the mechanism to minimize clearances around different size guidewires.

In any of the above-described embodiments, the brush of medical device 200, 500, 600, 800, 1000, 1100 may be replaced by a basket for stone retrieval, a stent or stent delivery system, an optical wand, forceps for biopsy, a retrieval device (e.g., such as a snare or other retrieval device), hemostasis device, or any other endoscopic tool known in the art. In some examples, medical device 200, 500, 600, 800, 1000, 1100 may include more than one tool, such as a brush and a snare device or a brush and stent delivery system.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A device, comprising:
a shaft having a brush at a distal portion of the shaft;
a distal coupler configured to removably couple to a guidewire, wherein the shaft is coupled to the distal coupler;
wherein the device is configured to slide in a distal direction or proximal direction along the guidewire.

2. The device of claim 1, wherein the shaft is configured to pivot relative to the distal coupler.

3. The device of claim 1, wherein the distal coupler includes a central channel, a proximal U-shaped portion, a distal U-shaped portion, and a slot between the proximal U-shaped portion and the distal U-shaped portion.

4. The device of claim 3, wherein the distal coupler further comprises: (i) a middle portion positioned between the proximal U-shaped portion and the distal U-shaped portion, (ii) a first gap positioned between the middle portion and the distal U-shaped portion, and (iii) a second gap positioned between the middle portion and the proximal U-shaped portion.

5. The device of claim 4, wherein the middle portion includes an extending portion extending towards the slot.

6. The device of claim 1, wherein the distal coupler is tapered from a proximal portion to a distal portion.

7. The device of claim 1, wherein the shaft is coupled to the distal coupler via a ring-shaped hinge coupler.

8. The device of claim 3, wherein the distal U-shaped portion and the proximal U-shaped portion are longitudinally aligned.

9. The device of claim 1, wherein the distal coupler is cylindrical and includes a central lumen extending longitudinally entirely through the distal coupler, wherein a channel extends an entire longitudinal length of the distal coupler, and wherein the channel provides a lateral opening into the central lumen.

10. The device of claim 9, wherein the channel extends between opposing surfaces of distal coupler, and the channel has a width configured to allow a guidewire pass through the channel.

11. The device of claim 10, wherein the channel extends transverse to a central longitudinal axis of the distal coupler.

12. The device of claim 10, wherein the channel extends helically around the distal coupler.

13. The device of claim 12, wherein the distal coupler is tapered and at least partially conical.

14. The device of claim 10, wherein the channel is longitudinally aligned with a central longitudinal axis of the distal coupler.

15. The device of claim 1, wherein the distal coupler includes a main body, a first curved extension, a second curved extension, and first gap between the first curved extension and the second curved extension; wherein the first curved extension forms a second gap between the first curved extension and the main body; wherein the second curved extension forms a third gap between the second curved extension and the main body; wherein the third gap opens into the first gap, wherein first gap opens into the second gap; and wherein each of the first gap, the second gap, and the third gap are sized to receive the guidewire.

16. The device of claim 1, wherein the distal coupler includes a first or proximal portion and a second or distal portion, and wherein the first proximal portion is removably coupled to the second distal portion via threads or grooves configured to receive threads.

17. The device of claim 16, wherein the distal coupler includes a proximal portion, a middle portion, a distal portion, and a hinge member extending between the proximal portion and the distal portion; wherein the hinge member is configured to snap-fit or otherwise removably couple to middle portion and/or proximal portion and/or distal portion.

18. A device, comprising:
a shaft; and
a distal coupler configured to removably couple to a guidewire, wherein the shaft is coupled to the distal coupler and the distal coupler comprises:
a central channel extending an entire longitudinal length of the distal coupler and including an opening facing a direction transverse from a central longitudinal axis of the distal coupler,
a proximal U-shaped portion,
a distal U-shaped portion,
a middle portion extending between the proximal U-shaped portion and the distal U-shaped portion, and
a hinge member rotatably coupled to at least one of the proximal U-shaped portion, the distal U-shaped portion, and the middle portion;
wherein the hinge member is configured to transition from: (i) a position outside of the opening to (ii) a position extending across the opening, wherein the hinge member is configured to snap-fit or otherwise removably couple to the middle portion and/or the proximal U-shaped portion and/or the distal U-shaped portion when positioned extending across the opening; and
wherein the device is configured to slide in a distal direction or proximal direction along the guidewire.

19. A device, comprising:
a shaft;
a distal coupler coupled to the shaft and configured to removably couple to a guidewire, wherein the distal coupler includes:
a central channel,
a proximal U-shaped portion,
a distal U-shaped portion,
a slot between the proximal U-shaped portion and the distal U-shaped portion,
a middle portion positioned between the proximal U-shaped portion and the distal U-shaped portion,
a first gap positioned between the middle portion and the distal U-shaped portion, and
a second gap positioned between the middle portion and the proximal U-shaped portion;
wherein the device is configured to slide in a distal direction or proximal direction along the guidewire.

20. The device of claim 19, wherein the middle portion includes an extending portion extending radially inward towards the slot.
